(19) United States
(12) Patent Application Publication
(10) Pub. No.: US 2026/0131090 A1
(43) Pub. Date: May 14, 2026

(54) DEVICE FOR DISPENSING A FLUID PRODUCT

(71) Applicant: APTAR FRANCE SAS, LE NEUBOURG (FR)

(72) Inventors: Guy LELOUP, SAINT AUBIN LES ELBEUF (FR); Franck POULLAIN, LA HAYE MALHERBE (FR)

(73) Assignee: APTAR FRANCE SAS, LE NEUBOURG (FR)

(21) Appl. No.: 19/119,481

(22) PCT Filed: Oct. 6, 2023
(86) PCT No.: PCT/FR2023/051556

§ 371 Date: Apr. 9, 2025

(30) Foreign Application Priority Data
Oct. 10, 2022 (FR) FR2210363

(51) Int. Cl. A61M 15/08; A61M 15/00

(52) U.S. Cl. CPC A61M 15/08; A61M 15/0041; A61M 2205/586

(57) ABSTRACT

A device for dispensing a fluid having a reservoir containing fluid and a dispensing head with a dispensing orifice and a radial flange forming a finger rest during actuation. The reservoir is axially movable relative to the dispensing head during actuation, a stopper/piston is movable in the reservoir to dispense fluid through the dispensing orifice during actuation. The device has a blocking member that blocks axial movement of the reservoir in the dispensing head, the blocking member being movable and/or deformable between a blocking position and a release position. The device has fins on or above the radial flange and movable between a rest position, in which the blocking member is in the blocking position, and a pressing position, in which the blocking member is moved and/or deformed into the release position. The blocking member includes a hollow axial sleeve assembled around the dispensing head, above the radial flange.

The present invention relates to a device for dispensing a fluid product. More specifically, the present invention relates to a unidose device for the nasal dispensing of fluid product to dispense a pharmaceutical fluid product in the nose of a user by way of one single nasal sprays.

A drawback of existing devices for nasal dispensing of fluid product is the risk of undesired actuation in the event of a fall of the device.

The present invention aims to provide a device for dispensing a fluid product which does not reproduce the above-mentioned drawbacks.

More particularly, an object of the present invention is to provide a device for dispensing a fluid product that avoids any risk of undesired actuation, for example in the event of a fall of the device.

Another object of the present invention is to provide such a device in which the risk of undesired actuation in the event of a fall is minimal or even zero.

Another object of the present invention is to provide such a device that is simple and inexpensive to manufacture and to assemble.

The present invention thus provides a device for dispensing a fluid product comprising a reservoir containing fluid product and a dispensing head that is provided with a dispensing orifice and a radial flange that forms a finger rest during actuation, said reservoir being axially movable relative to said dispensing head during actuation, a stopper/piston being movable in said reservoir so as to dispense fluid product through said dispensing orifice during actuation, said device comprising a blocking member that blocks the axial movement of said reservoir in said dispensing head, said blocking member being movable and/or deformable between a blocking position and a release position, said device comprising fins arranged on or above said radial flange, said fins being movable between a rest position, in which said blocking member is in the blocking position, and a pressing position, in which said blocking member is moved and/or deformed into the release position, said blocking member including a hollow axial sleeve that is assembled around said dispensing head, above said radial flange.

Advantageously, said reservoir is formed by a body that is hollow and blind, comprising a bottom and a single opening which, in the rest position, is sealed by said stopper/piston.

Advantageously, the device comprises a hollow insert inserted and fixed in said dispensing head, said insert supporting a piercing tip to pierce said stopper/piston and thus connect said reservoir to said dispensing orifice during actuation.

Advantageously, said piercing tip is made separately and then assembled in said insert, in particular by force-fitting or by overmoulding.

In an advantageous variant, said piercing tip is moulded integrally with said insert.

Advantageously, said hollow axial sleeve comprises at least one axial extension, advantageously two diametrically opposite axial extensions, extending axially upwards and each provided with a radial projection extending radially inwards, each axial extension being associated with a respective fin that extends radially outwards from said hollow axial sleeve.

Advantageously, each axial extension forms with its associated fin an assembly approximately in the shape of a V and substantially rigid.

Advantageously, said dispensing head comprises at least one lateral opening, advantageously two diametrically opposed lateral openings, each adapted to engage with a radial projection of said blocking member such that in the blocking position of said blocking member, each radial projection passes through a lateral opening and is positioned between an upper edge of an actuation body receiving said reservoir and said neck of said reservoir, such that in the blocking position of said blocking member, an upward axial thrust on said actuation body is blocked by said radial projections, preventing said actuation body, and therefore said reservoir, from moving axially in said dispensing head.

Advantageously, the device includes an actuation body receiving said reservoir, said actuation body including a radial disk connected to said actuation body by one or more breakable bridges, said radial disk engaging with a portion of said dispensing head so as to form a precompression threshold that the user must overcome during actuation so as to break said breakable bridges and thus actuate the device.

Advantageously, said reservoir contains one single dose of fluid product dispensed in one single actuation.

These and other characteristics and advantages of the present invention appear more clearly from the following detailed description, given by way of non-limiting examples, and with reference to the accompanying drawings, and in which:

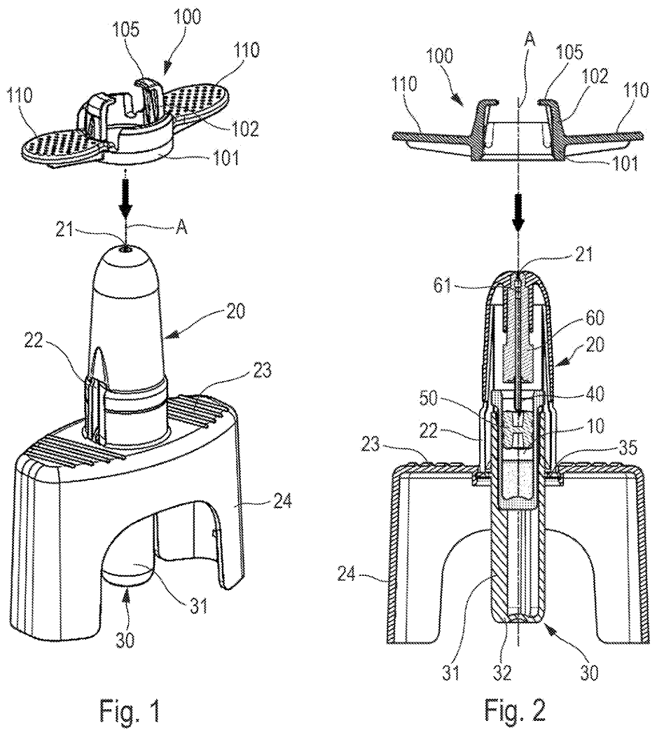

FIG. 1 is a schematic, perspective view of a device for dispensing fluid product according to a advantageous embodiment, before assembly of the blocking member,

FIG. 2 is a diagrammatic section view of the FIG. 1 device;

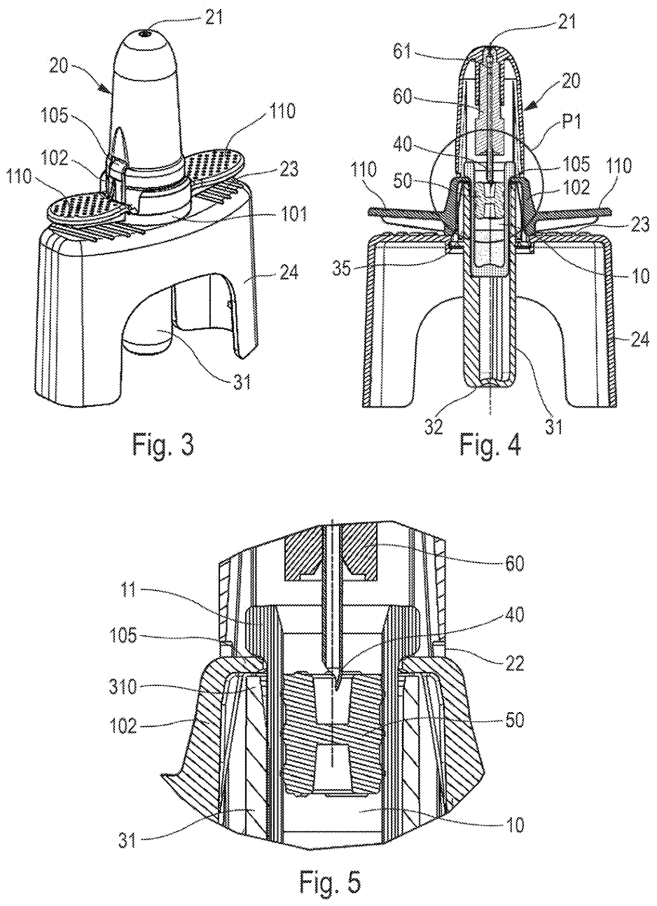

FIG. 3 is a view similar to the view in FIG. 1, in position after assembly of the blocking member and before actuation thereof;

FIG. 4 is a diagrammatic section view of the FIG. 3 device;

FIG. 5 is an enlarged view of the part P1 of FIG. 4,

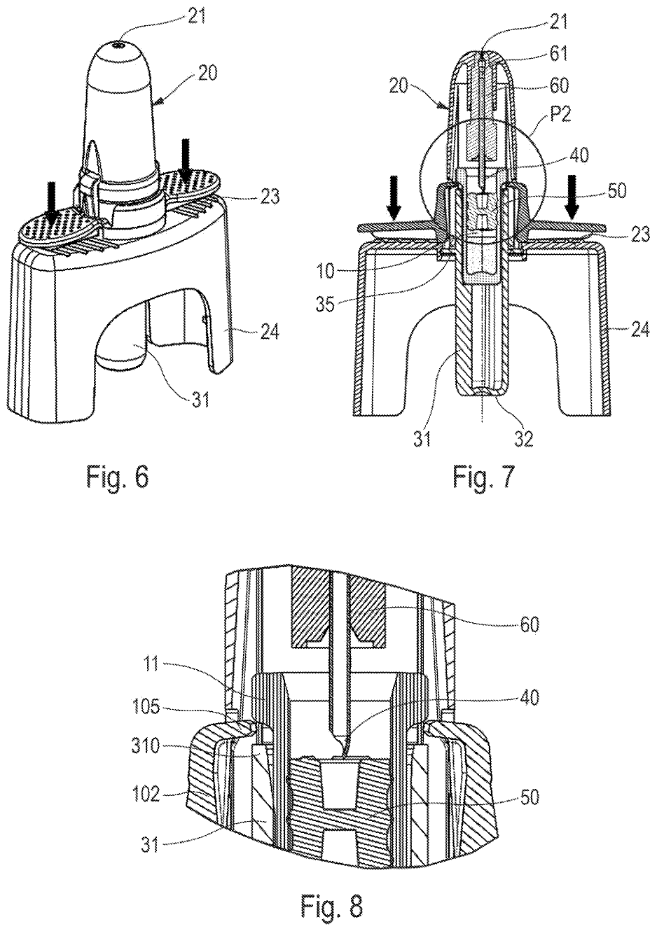

FIG. 6 is a view similar to that of FIG. 3, in position after actuation of the blocking member and with the piston in the rest position,

FIG. 7 is a diagrammatic section view of the FIG. 6 device;

FIG. 8 is an enlarged view of the part P2 of FIG. 7,

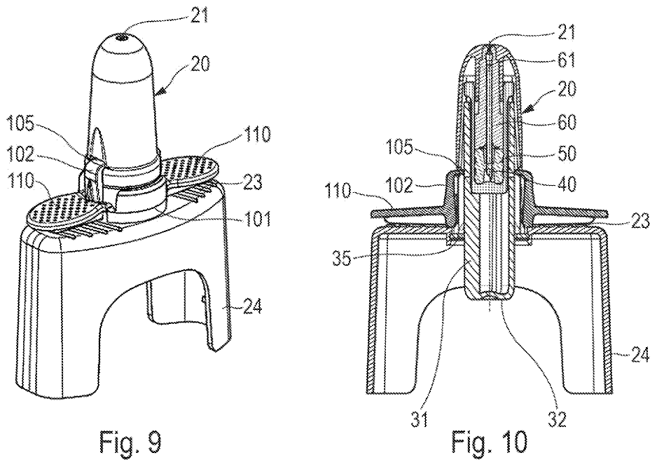

FIG. 9 is a view similar to the view in FIG. 6, after actuation of the piston; and

FIG. 10 is a diagrammatic section view of the FIG. 9 device;

The terms “axial” and “radial” are relative to the longitudinal central axis A of the device. The terms “top” and “bottom” are relative to the upright position of the device shown in Figures.

In reference to the Figures, a preferred embodiment of the present invention is represented. In this embodiment, the device is a unidose device, that is to say that the reservoir contains a single dose of fluid product dispensed in one single actuation.

In this embodiment, a reservoir 10 containing fluid product to be dispensed, typically a liquid, is arranged inside a body that forms a dispenser head 20. The dispensing head 20 comprises a dispensing orifice 21 oriented axially. The dispensing orifice 21 serves to dispense a dose of fluid out from said dispensing head 20 while the device is being actuated by a user.

The dispensing head 20 comprises an extended nosepiece comprising at its proximal axial end, said dispensing hole 21, as well as a lateral body 24, connected to said nosepiece 22 at a radial flange 23.

In the example of FIG. 1, a hollow insert 60 is arranged in the dispensing head 20, upstream from said dispensing orifice 21, said hollow insert 60 defining a central expulsion channel and, in engagement with the bottom wall of the dispensing head 20, a spraying profile (not visible in the drawings) directly upstream from said dispensing orifice 21.

The channel 61 terminates at its bottom end in a piercing tip 40. This piercing tip 40 may be moulded integrally with the insert 60. As a variant, it could be made separately, for example out of metal or of a synthetic material other than that of the insert, and then assembled in said insert, for example by force-fitting or by overmoulding. The piercing tip 40 extends axially downwards beyond the elongated cylindrical body of the insert 60.

The reservoir 10 is advantageously formed by a blind hollow body, comprising one single opening defining a neck 11, and closed by a stopper/piston 50. In the example represented, which is a unidose, the reservoir 10 contains a single fluid product dose to dispense with one single actuation of the device. The stopper/piston 50 can be displaced in the reservoir 10 between a rest position and an actuation end position to dispense said fluid product through the dispensing orifice 21 during the actuation.

Actuation means 30 are advantageously provided so as to make it possible to actuate the device.

In the embodiment of FIG. 1, the actuation means 30 comprise an actuation body 31 that is movable relative to the dispensing head 20, said actuation body 31 engaging with said reservoir 10 so as to move it axially relative to the dispensing head 20, towards the dispensing orifice 21.

To actuate the device, the user presses on the bottom wall 32 of the actuation body 31, to move the latter together with the reservoir 10 axially in the dispensing head 20, thus causing the piercing tip 40 to pierce the stopper/piston 50 and the dose of fluid to be dispensed through the dispensing orifice 21. In the absence of an actuation body 31, the user could press directly on the bottom of the reservoir 10. During such actuation, the reservoir 10 is thus pushed axially towards the dispensing orifice 21, so that the piercing tip 40 pierces the stopper/piston 50. The contents of the reservoir 10 are thus connected to the dispensing orifice 21, and the user pressing on the actuation body 31 moves the stopper 50 in the reservoir 10 so as to deliver the fluid product.

It should be noted that the present invention could also apply to devices that do not have a piercing tip suitable for piercing a stopper/piston, as described, for example, in documents WO2019122671, WO2021069823, WO2022090675, WO2022101589, WO2022208014.

Advantageously, the actuation body 31 includes a radial disk 35 connected to the actuation body by one or more breakable bridges 36, that can be seen in particular in FIG. 17. The disk 35 engages with a portion of the dispensing head 20 to form a precompression threshold that the user must overcome during actuation in order to break the breakable bridges 36 and thus actuate the device. These breakable bridges 36 provide safety against undesired actuation, but during an axial fall of the device, it is not excluded that the inertia of the device is sufficient to break the breakable bridges and thus the safety provided by the breakable bridges 36 is not sufficient.

According to the invention, the device includes a blocking member 100 which blocks the axial movement of the reservoir 10 in the dispensing head 20. The blocking member 100, 200 is movable and/or deformable between a blocking position and a release position. The user must first move and/or deform the blocking member 100, to its release position before to be able to actuate the device by axially moving the reservoir 10 in the dispensing head 20.

According to the invention, the device comprises fins 110 arranged on or above the radial flange 23, said fins being movable between a rest position, in which the blocking member 100 is in the blocking position, and a bearing position, in which the blocking member 100 is moved and/or deformed into the release position.

Advantageously, the invention provides that the movement and/or the deformation of the blocking member 100 towards its release position is achieved when the user takes the device in hand, by pressing his fingers on the radial flange 23 of the dispensing head 20. Thus, the present invention makes it possible to guarantee safety in the event of a fall of the device, without requiring an additional movement of the user to actuate the device. When the user takes the device in hand, he places two fingers on the fins 110 arranged on or above the radial flange 23 of the dispensing head 20, and presses with the thumb on a distal axial bottom 32 of said actuation body 31. By pressing to actuate the device, he will first move with his fingers the fins 110, 210 towards their bearing position, which moves and/or deforms the blocking member 100 towards its release position, and then the thumb will move the actuation body 31 to dispense the dose.

According to the invention, the blocking member 100 is assembled around the dispensing head 20, above the radial flange 23.

The blocking member 100 comprises a hollow axial sleeve 101 which is assembled, in particular with friction, on the dispensing head 20.

The axial hollow sleeve 101 includes at least one axial extension 102, advantageously two diametrically opposite axial extensions 102, extending axially upwards and each provided with a radial projection 105 extending radially inwards.

Each axial extension 102 is associated with a respective fin 110 that extends radially outwards from the hollow axial sleeve 101. In the example shown, there are therefore two diametrically opposed fins 110 which, when the blocking member 100 is assembled on the dispensing head 20, are positioned above the radial flange 23 forming a finger rest. Thus, when the user takes the device in their hand, their fingers are no longer placed on the radial flange 23 but on the fins 110. Advantageously, each axial extension 102 forms with its associated fin 110 an assembly approximately in the shape of V and substantially rigid.

The dispensing head 20 comprises at least one lateral opening 22, advantageously two diametrically opposed lateral openings 22, each adapted to engage with a radial projection 105 of the blocking member 100 when the latter is assembled on the dispensing head 20.

As can be seen in FIG. 5 in particular, in the blocking position of the blocking member 100, each radial projection 105 passes through a lateral opening 22 in order to be positioned between an upper edge 310 of the actuating body 31 and the neck 11 of the reservoir 10. Thus, in this blocking position, an upward axial thrust on the actuation body 31 is blocked by the radial projections 105, preventing axial displacement of said actuation body 31, and thus of the reservoir 10, in the dispensing head 20. Any risk of accidental actuation is therefore prevented.

When the user presses on the fins 110, they pivot slightly downwards relative to the hollow axial sleeve 101, thus causing the axial extensions 102 to pivot outwards. This moves the radial projections 105 radially outwards so that they no longer block the actuation body 31, as can be seen in particular in FIG. 8. Thus, a pressure on the bottom 32 of the actuating body 31 will then move the latter axially upwards to dispense the dose of fluid, possibly after having broken the breakable bridges 36.

Thus, in order to actuate the device, it is necessary to simultaneously press axially on the fins 110 and on the bottom of the actuation body 31, which is impossible during an accidental fall of the device. The present invention therefore ensures perfect safety against undesired actuation in case of a fall of the device.

An advantage of the present invention is that it does not require additional movement or displacement of a portion of the device: the user simply takes the device in hand and performs the conventional actuation movement. Its fingers on the fins 110 will first move them and thus unlock the blocking member, then the thumb will move the reservoir 10 in the dispensing head 20 to dispense the dose of fluid.

Naturally, other variant embodiments may also be envisaged, without going beyond the scope of the present invention, as defined by the accompanying claims.

1. A device for dispensing a fluid product comprising a reservoir containing fluid product and a dispensing head that is provided with a dispensing orifice and a radial flange that forms a finger rest during actuation, said reservoir being axially movable relative to said dispensing head during actuation, a stopper/piston being movable in said reservoir so as to dispense fluid product through said dispensing orifice during actuation, said device comprising a blocking member that blocks the axial movement of said reservoir in said dispensing head, said blocking member being movable and/or deformable between a blocking position and a release position, said device comprising fins arranged on or above said radial flange, said fins being movable between a rest position, in which said blocking member is in the blocking position, and a pressing position, in which said blocking member is moved and/or deformed into the release position, characterised in that said blocking member comprises a hollow axial sleeve that is assembled around said dispensing head, above said radial flange.

2. The device according to claim 1, wherein said reservoir is formed by a hollow and blind body, comprising a bottom and a single opening which, in the rest position, is sealed by said stopper/piston.

3. The device according to claim 2, comprising a hollow insert inserted and fixed in said dispensing head, said insert supporting a piercing tip to pierce said stopper/piston and thus connect said reservoir to said dispensing orifice during actuation.

4. The device according to claim 3, wherein said piercing tip is made separately and then assembled in said insert, in particular by force-fitting or by overmoulding.

5. The device according to claim 3, wherein said piercing tip is moulded integrally with said insert.

6. The device according to claim 1, wherein said hollow axial sleeve comprises at least one axial extension, advantageously two diametrically opposite axial extensions, extending axially upwards and each provided with a radial projection extending radially inwards, each axial extension being associated with a respective fin that extends radially outwards from said hollow axial sleeve.

7. The device according to claim 6, wherein each axial extension forms with its associated fin an assembly approximately in the shape of a V and substantially rigid.

8. The device according to claim 6, wherein said dispensing head comprises at least one lateral opening, advantageously two diametrically opposed lateral openings, each adapted to engage with a radial projection of said blocking member such that in the blocking position of said blocking member, each radial projection passes through a lateral opening and is positioned between an upper edge of an actuation body receiving said reservoir and said neck of said reservoir, such that in the blocking position of said blocking member, an upward axial thrust on said actuation body is blocked by said at least one radial projection, preventing said actuation body and therefore said reservoir, from moving axially in said dispensing head.

9. The device according to claim 1, comprising an actuation body receiving said reservoir, said actuation body comprising a radial disc connected to said actuation body by one or more breakable bridges, said radial disc engaging with a portion of said dispensing head to form a precompression threshold that the user must overcome during actuation to break said breakable bridges and thus actuate the device.

10. The device according to claim 1, wherein said reservoir contains a single fluid product dose dispensed in a single actuation.
